(19) United States
(12) Patent Application Publication
(10) Pub. No.: US 2021/0236066 A1
(43) Pub. Date: Aug. 5, 2021

(54) REMOTE CAREGIVER SYSTEM

(71) Applicant: Kelsey Wright, Hernando, MS (US)

(72) Inventor: Kelsey Wright, Hernando, MS (US)

(21) Appl. No.: 16/780,918

(22) Filed: Feb. 4, 2020

(51) Int. Cl. A61B 5/00; A61B 5/024; G16H 40/67; G16H 20/10

(52) U.S. Cl. CPC A61B 5/7465; A61B 5/024; G16H 20/10; A61B 5/7455; G16H 40/67; A61B 5/681

(57) ABSTRACT

A remote caregiver system including a wearable caregiver companion device and a caregiver processing platform. The caregiver companion device includes a band to be worn by a wearer. The band includes a plurality of buttons for actuation by the wearer to assist the wearer with daily life activities. A caregiver may operate a computing device, such as a smart phone or laptop, to remotely interact with the individual via the caregiver companion device.

### FIELD OF THE INVENTION

Embodiments described herein generally relate to caregiver systems, and more particularly to a remote caregiver system.

### BACKGROUND OF THE INVENTION

Monitoring the elderly is a standard practice throughout much of the world as this population is at an increased risk of injury and/or death. However, most elderly persons seek to maintain their independence and do not want to feel supervised by their younger counterparts yet still may require access to emergency services. Hence it is desirable to provide a way to assist the elderly in everyday living while still allowing them to maintain their independence.

### BRIEF DESCRIPTION OF THE DRAWINGS

The various advantages of the embodiments of the present disclosure will become apparent to one skilled in the art by reading the following specification and appended claims, and by referencing the following drawing(s), in which:

FIG. 1 shows an exemplary view of a remote caregiver system according to an embodiment of the present disclosure.

FIG. 2 shows an exemplary view of a living assistant wearable device according to an embodiment of the present disclosure.

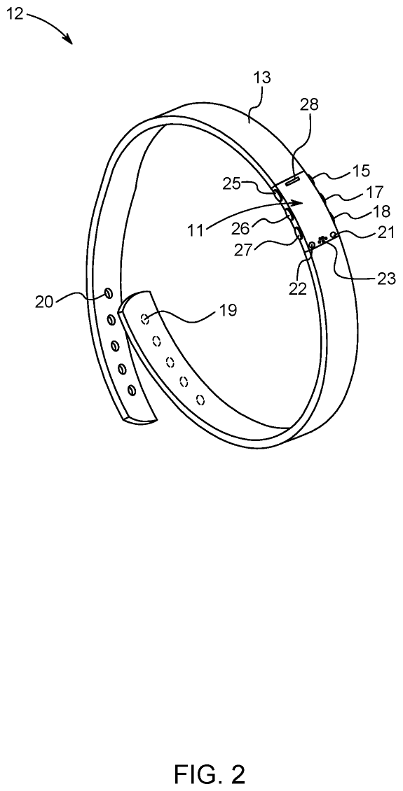

### SUMMARY OF THE INVENTION

Exemplary embodiments disclosed herein describe a remote caregiver system. The system includes at least one wearable caregiver companion, a network engine and at least one caregiver processing platform. The at least one caregiver companion devices includes a band configured to be worn around a wearer's wrist. The band includes a first strap member and a second strap member. The first strap member includes a plurality of apertures, the second strap member includes a plurality of snap elements. The plurality of apertures and snap elements are attached to one another to securely attach the band to the wearer's wrist. Further, the caregiver companion device includes a controller chip attached to the band for transmitting and receiving signals, the controller chip including at least one processor; at least one sensor for monitoring a vital sign; a network interface for interfacing with a network engine; and a plurality of user interface buttons, wherein each user interface button corresponds to a caregiver service provided by a caregiver processing platform. The at least one caregiver processing platform includes at least one processor; and at least one memory for storing executable instructions, the processor configured to execute the instructions to continuously monitor and process data received from the caregiver companion device; generate content relating to caregiver services to transmit to the caregiver companion device; and to transmit the generated content to the wearable caregiver companion device.

In some exemplary embodiments, the wearable caregiver companion device further comprises a speaker for providing audible messages.

In some exemplary embodiments, the actuation of any of the plurality of user interface buttons causes an audible message to be played.

In some exemplary embodiments, at least one or more of the plurality of user interface buttons correspond to reminders-notifications to the wearer.

In some exemplary embodiments, at least one or more of the plurality of user interface buttons correspond to safety features.

In some exemplary embodiments, the wearable caregiver companion device further comprises a vibrating mechanism to notify the wearer when an incoming message has been received.

In some exemplary embodiments, the at least one sensor monitors the wearer's heart rate.

In some exemplary embodiments, the controller chip sends a message to the caregiver processing platform if the wearer heart rate is detected to be out of range.

### DETAILED DESCRIPTION

The disclosure will be described more fully hereinafter with reference to the accompanying drawings, in which exemplary embodiments of the disclosure are shown. This disclosure may, however, be embodied in many different forms and should not be construed as limited to the exemplary embodiments set forth herein. It will be apparent to persons skilled in the relevant art that various changes in form and detail can be made to various embodiments without departing from the spirit and scope of the present disclosure. Thus, the breadth and scope of the present disclosure should not be limited by any of the above-described exemplary embodiments but should be defined only in accordance with the following claims and their equivalents.

The present disclosure relates to a remote caregiver system (“the system”). The system allows a caregiver to remotely provide caregiver services to assist an individual with activities of daily living. The caregiver services may include providing medication reminders, appointment reminders, daily health management, monitoring safety issues, contacting a third party (e.g., ambulance, 911, doctor) to assist the wearer when a safety issue arises, etc. The individual may be elderly or ill or simply not in a position to take care of themselves and may require assistance. A caregiver may operate a computing device, such as a smart phone or laptop, which is synched with a caregiver companion device to facilitate interaction between an individual and a caregiver. The individual wears the companion device and the caregiver companion device interacts with the caregiver's computing device using Bluetooth or over the Internet using WIFI.

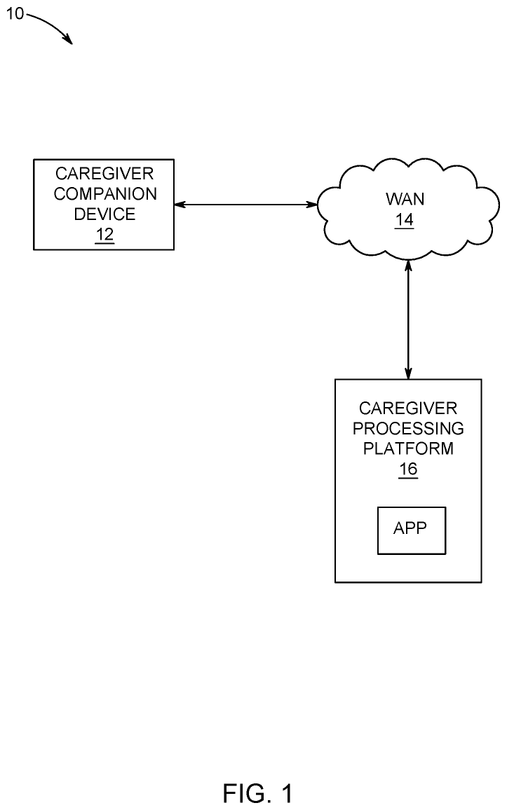

As illustrated in FIG. 1, the system 10 includes at least one caregiver companion device 12, network engine (i.e., wide area network (WAN)) 14, and at least one caregiver processing platform 16. The caregiver companion device includes a band 13 intended to be worn around a wearer's wrist. The band 13 may be made of any water-proof material. For discretion and for easy concealment, the band may have a thin body (i.e., the width should be in the range of 0.5″-0.7″) and a small face (i.e., the length should be in the range of 1.4″-1.7″). The band may be of any color. In a preferred embodiment, the band is black.

The band includes a first strap member and a second strap member. The first strap member includes a plurality of apertures 18 and the second strap member includes a plurality of snap elements 19 which are configured to attached to the plurality of apertures. The size of the band may be adjusted by attaching more or less of the plurality of snap elements to the plurality of apertures. Once the snap elements 19 and the apertures 18 are attached, the band 13 is securely attached to the wearer's wrist. The band 13 includes a housing unit 11 positioned at the center of the band. The first strap member and the second strap member are positioned at opposite ends of the housing unit. The housing unit stores/encloses other components of the caregiver companion device 12. The housing unit includes the small face and a plurality of buttons attached at each side of the housing.

Further, the caregiver companion device 12 includes a controller chip (located inside housing unit 11), at least one sensor (located inside housing unit 11), a network interface (i.e., the hardware that connects the device 12 to the wide area network (i.e., 14) and that connects/pairs the device with Bluetooth—located inside housing unit 11), a speaker (23), a vibrating mechanism (located inside housing unit 11), a battery charging element (located inside housing unit 11), a plurality of user interface buttons (located along the sides of the housing unit 11) such as, medication reminder button 15, appointment reminder button 17, heart rate button 18, date and time button 25, alerts button 26 and help button 27), and a plurality of user operating buttons, such as, WIFI button 21, Bluetooth button 22, and vibrating mode switch 28.

The controller chip transmits and receives signals and processes data. The controller chip includes at least one processor. The at least one sensor may monitor a vital sign of the wearer, such as, for example, heart rate, blood pressure, etc. The caregiver companion device 12 may include a heart rate sensor for monitoring the wearer's heart rate. When the device detects a heart rate that is abnormal (i.e., not in a healthy range for the wearer), the controller chip sends an alert to the caregiver processing platform 16.

In another exemplary embodiment, the caregiver companion device 12 includes a fall detection sensor for detecting when the wearer has fallen. The controller chips sends an alert to the caregiver processing platform when a fall is detected. In another exemplary embodiment, the caregiver companion device includes a motion sensor for determining if the wearer has been sedentary. The controller chip would cause a message (e.g., telling the wearer to stand) to be spoken to the wearer if it was determined that the wearer has been sedentary.

The speaker 23 may include any suitable speaker. The caregiver companion device 12 uses the speaker as the primary form of communication with the wearer. Some of the intended wearers of the device 12 are elderly people and blind individuals, both of which may find text messages difficult/impossible to read. In a preferred embodiment, the speaker is a loud speaker. This allows effective use of the device by individuals who have hearing challenges, such as elderly people. However, in some alternative embodiments, the caregiver companion device may include a display screen for displaying information.

The vibrating mechanism vibrates the caregiver companion device 12 to notify the wearer that an incoming message has been received. The vibrating mechanism may be implemented using any suitable vibrators, such as a vibrating motor. Once the device 12 vibrates, the speaker will announce a message to the wearer, such as, for example, “new medication added”, or “new alert added”, and the wearer can retrieve the incoming message by depressing the corresponding user interface button on the housing unit.

The caregiver companion device 12 includes a battery charging element for charging the device. Any suitable charging element may be used. In a preferred embodiment, the charging element is a magnetic charger located on the backside of the housing unit which may be charged with a charging pad.

In another alternative embodiment, the caregiver companion device 12 may include a camera for capturing a real time image of the wearer. The controller chip may transmit the image to the caregiver processing platform 16.

The caregiver companion device 12 includes a plurality of user interface buttons which each correspond to a caregiver service (e.g., reminders/notifications/safety measures) provided by a caregiver via caregiver processing platform 16. The plurality of user interface buttons may be implemented in different colors. Alternatively, the plurality of user interface buttons are black. At least half of the plurality of user interface buttons are each located on the right side and the left side respectively of the housing unit at predefined positions, such as, for example, position 1, position 2, position 3, position 4, position 5 and position 6, etc.

As noted, the plurality of user interface buttons may include medication reminder button 15, appointment reminder button 17, heart rate button 18, date and time button 25, alerts button 26 and help button 27. The medication reminder button when depressed will cause an audio message to be played, such as, for example, “take your aspirin at 9 am.” The appointment reminder button when depressed will cause an audio message to be played, such as, for example, “you have a doctor's appointment tomorrow at 10 am.” The heart rate button when depressed will cause an audio message to be played, such as, for example, “your heart rate is 60 bpm”. The date and time button when depressed will cause an audio message to be played indicating the current date and time, such as, “today is January 1st and the time is 9 am.” The alerts button when depressed will cause one or more messages sent by the caregiver to be plated, such as, for example, “don't forget to lock the door”. The help button when depressed for ten (10) seconds will send an alert to the caregiver indicating that the individual needs assistance.

The wearer may place the device in silent mode by depressing the date and time button for ten seconds. In the silent mode, audio messages are not played. By default, after two hours the device returns to its default state of playing audio messages. When the device resumes active mode, the device 12 will alert the wearer of any updated or incoming messages that were received while the device was in silent mode.

In operation, during setup of the caregiver companion device 12, audio instructions will play from the speakers informing the wearer how to use the device 12. The plurality of user interface buttons will be explained and the position of each user interface button on the housing unit will be explained, such as, for example, medication reminder button is located at position 1, appointment reminder is located at position two, and heart rate button is located at position three. The positions of the buttons will be explained, such as, for example, position one (1) is the first button located on the top left side of the housing unit. Position two (2) is the button immediately below position one and position three (3) is the button immediately below position two.

In another alternative embodiment the caregiver companion device 12 may include a plurality of user operating buttons, such as, WIFI button 21, Bluetooth button 22, and vibrating mode switch 28. The WIFI button may be depressed to manually activate a WIFI connection. The Bluetooth button 22 may be depressed to manually activate a Bluetooth connection with another device. The vibrating mode switch 28 may be manually activated to place the caregiver companion device 12 into vibrate mode.

The network engine 14 may include any one of or a combination of multiple different types of networks, such as, cable networks, the Internet, wireless networks, and other private and/or public networks. In some instances, the network engine 14 may include cellular, Wi-Fi, or Wi-Fi direct.

The caregiver processing platform 16 includes at least one processor and at least one memory for storing executable instructions. The processor is configured to execute the instructions to continuously monitor and process data received from the caregiver companion device, to generate content relating to caregiver services to transmit to the caregiver companion device and to transmit the content to the caregiver companion device.

The memory can include anyone or combination of volatile memory elements (e.g., random access memory (RAM, such as DRAM, SRAM, SDRAM, etc.)) and nonvolatile memory elements (e.g., ROM, hard drive, tape, CDROM, etc.). Moreover, the memory device may incorporate electronic, magnetic, optical, and/or other types of storage media. In the context of this document, a “non-transitory computer-readable medium” can be, for example but not limited to, an electronic, magnetic, optical, electromagnetic, infrared, or semiconductor system, apparatus, or device. More specific examples (a non-exhaustive list) of the computer-readable medium would include the following: a portable computer diskette (magnetic), a random access memory (RAM) (electronic), a read-only memory (ROM) (electronic), an erasable programmable read-only memory (EPROM, EEPROM, or Flash memory) (electronic), and a portable compact disc read-only memory (CD ROM) (optical). The processor may include one or more of microprocessors, graphics processors, coprocessors, etc.

A caregiver may remotely interact with the individual by synching a computing processing device, such as, for example, a laptop, tablet or smart phone to the caregiver companion device 12. The caregiver must first download a software application (i.e., computer executable instructions) to the computing processing device to perform the synching. Once downloaded, the processor may synch to the caregiver companion device 12 using the software application, WIFI and/or Bluetooth. The software application prompts the caregiver to input medication reminders, appointment reminders, heart rate parameters, the date and time, and any other notifications that the caregiver would like to provide to the individual (wearer). The inputted information is transmitted to the caregiver companion device and stored therein. As noted, by depressing one of the plurality of user interfaces buttons, an audio message corresponding to the associated information will be played to the user. The caregiver can continuously interact with the caregiver companion device 12 and the caregiver may provide updates at any time.

Generally, computer-executable instructions form software component(s) which may include routines, programs, objects, components, data structures, and the like that perform particular functions or implement particular abstract data types. The software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

A software component (including a software application) may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Moreover, it should be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.)

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises,” “comprising,” “includes” and/or “including,” when used herein, specify the presence of stated features, integers, steps, operations, elements and/or components, but do not preclude the presence or addition of one or more other features, integers, steps, operations, elements, components and/or groups thereof.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art. However, should the present disclosure give a specific meaning to a term deviating from a meaning commonly understood by one of ordinary skill, this meaning is to be taken into account in the specific context this definition is given herein.

Those skilled in the art will appreciate from the foregoing description that the broad techniques of the embodiments of the present invention may be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

What is claimed is:

1. A remote caregiver system comprising:
at least one wearable caregiver companion device comprising:
a band configured to be worn around a wearer's wrist, the band including a first strap member and a second strap member, the first strap member including a plurality of apertures, the second strap member including a plurality of snap elements, wherein the plurality of apertures and snap elements are attached to one another to securely attach the band to the wearer's wrist;
a controller chip attached to the band for transmitting and receiving signals, the controller chip including at least one processor;
at least one sensor for monitoring a vital sign;
a network interface for interfacing with a network engine; and
a plurality of user interface buttons, wherein each user interface button corresponds to a caregiver service provided by a caregiver processing platform;
a network engine; and
at least one caregiver processing platform comprising:
at least one processor;
at least one memory for storing executable instructions, the processor configured to execute the instructions to:
continuously monitor and process data received from the caregiver companion device;
generate content relating to caregiver services to transmit to the caregiver companion device; and
transmit the generated content to the wearable caregiver companion device.

2. The system of claim 1, wherein the wearable caregiver companion device further comprises a speaker for providing audible messages.

3. The system of claim 1, wherein actuation of any of the plurality of user interface buttons causes an audible message to be played.

4. The system of claim 1, wherein at least one or more of the plurality of user interface buttons correspond to reminders-notifications to the wearer.

5. The system of claim 1, wherein at least one or more of the plurality of user interface buttons correspond to safety features.

6. The system of claim 1, wherein the wearable caregiver companion device further comprises a vibrating mechanism to notify the wearer when an incoming message has been received.

7. The system of claim 1, wherein the at least one sensor monitors the wearer's heart rate.

8. The system of claim 1, wherein the controller chip sends a message to the caregiver processing platform if the wearer's heart rate is detected to be out of range.

9. A wearable caregiver companion device comprising:
a band configured to be worn around a wearer's wrist, the band including a first strap member and a second strap member, the first strap member including a plurality of apertures, the second strap member including a plurality of snap elements, wherein the plurality of apertures and snap elements allow the size of the band to be adjusted by attaching one or more of the snap elements to one or more of the apertures, the band is securely attached to the wearer's wrist when the snap elements and apertures are attached;
a controller chip attached to the band for transmitting and receiving signals, the controller chip including at least one processor;
at least one sensor for monitoring a vital sign;
a network interface for interfacing with a network engine; and
a plurality of user interface buttons, wherein each user interface button corresponds to a caregiver service provided by a caregiver processing platform which each correspond to a caregiver service provided by a caregiver processing platform.

10. The device of claim 9, further comprising a speaker for providing audio messages to the wearer.

11. The device of claim 9, further comprising a vibrating mechanism to notify the wearer when an incoming message has been received.

12. The device of claim 9, wherein actuation of any of the plurality of user interface buttons causes an audible message to be played.

13. The device of claim 9, wherein at least one or more of the plurality of user interface buttons correspond to reminders-notifications to the wearer.

14. The device of claim 9, wherein at least one or more of the plurality of user interface buttons correspond to safety features.

15. The device of claim 14, wherein a reminder-notification includes a medication reminder.

16. The device of claim 14, wherein a reminder-notification includes an appointment reminder.

17. The device of claim 9, wherein the at least one sensor monitors the wearer's heart rate.

18. The device of claim 9, wherein the controller chip sends a message to a caregiver if the wearer's heart rate is detected to be out of range.
